(19) United States
(12) Patent Application Publication
(10) Pub. No.: US 2026/0198934 A1
(43) Pub. Date: Jul. 16, 2026

(54) APPARATUS FOR FORMING A CANAL IN BONE MEDULLARY CAVITY

(71) Applicant: T.A.G. Medical Products Corporation Ltd., Kibbutz Gaaton (IL)

(72) Inventors: Idan BOADER, Karmiel (IL); Inbal HUVEN, Haifa (IL); Hagay BOTANSKY, Haifa (IL); Uri BARAK, Nesher (IL)

(73) Assignee: T.A.G. Medical Products Corporation Ltd., Kibbutz Gaaton (IL)

(21) Appl. No.: 19/137,665

(22) PCT Filed: Dec. 6, 2023
(86) PCT No.: PCT/IL2023/051249

§ 371 Date: Jun. 11, 2025

Related U.S. Application Data
(60) Provisional application No. 63/431,704, filed on Dec. 11, 2022.

(51) Int. Cl. A61B 17/16; A61B 17/56

(52) U.S. Cl. CPC A61B 17/164; A61B 17/1617; A61B 2017/564

(57) ABSTRACT

Disclosed herein is an apparatus for forming a canal in a medullary cavity of a bone, comprising a shaft having a proximal portion and a distal portion, an actuator configured to be moveable relative to the shaft, and a laterally extendable assembly operatively coupled to the distal portion of the shaft. The laterally extendable assembly is configured to transition between retracted and extended states in a lateral axis perpendicular to a longitudinal axis of the shaft. Wherein a movement of the actuator relative to the shaft induces transition of the laterally extendable assembly between the retracted and extended states to adjust an external diameter of the laterally extendable assembly.

### RELATED APPLICATION/S

This application claims the benefit of priority of U.S. Provisional Patent Application No. 63/431,704, filed on Dec. 11, 2022, the contents of which are incorporated herein by reference in their entirety.

### FIELD AND BACKGROUND OF THE INVENTION

The present invention, in some embodiments thereof, relates to an apparatus for forming a canal in bone medullary cavity and, more particularly, but not exclusively, to an apparatus having an adjustable diameter for forming a canal in bone medullary cavity.

The constant and rapid evolution of medical technology has introduced major advancements in numerous medical and surgical procedures targeting practically any organ, system, tissue and/or cell of the human body.

One of these rapid advancements are made in the field of arthroplasty which is directed for surgical replacement of joints, for example, shoulder, hip, knee, and/or the like. During such surgical procedures, a damaged joint and/or part thereof may be replaced with an artificial joint prosthesis.

The artificial joint prosthesis which may be made of metal, ceramic, heavy-duty plastic, and/or may look and move just like the natural joint it replaced.

### SUMMARY OF THE INVENTION

According to a first aspect of the present invention there is provided an apparatus for forming a canal in a medullary cavity of a bone, comprising a shaft having a proximal portion and a distal portion, an actuator configured to be moveable relative to the shaft, and a laterally extendable assembly operatively coupled to the distal portion of the shaft. The laterally extendable assembly is configured to transition between retracted and extended states in a lateral axis perpendicular to a longitudinal axis of the shaft, wherein a movement of the actuator relative to the shaft induces transition of the laterally extendable assembly between the retracted and extended states to adjust an external diameter of the laterally extendable assembly.

According to a second aspect of the present invention there is provided a method of forming a medullary canal in a bone, comprising configuring an external diameter of a laterally extendable assembly of a medullary canal forming apparatus to a minimal value, the external diameter is defined by an actuator moveable relative to a shaft of the apparatus for transitioning the laterally extendable assembly between a retracted and extended states, inserting a distal portion of the apparatus into a guide hole created in a medullary cavity of a bone to form a canal in the bone, increasing the external diameter by moving the actuator relative to the shaft to extend the laterally extendable assembly, rotating the shaft to increase a dimeter of the canal, gradually increasing the external diameter and rotating the shaft to increase the diameter of the canal until reaching a desired diameter, reducing the external diameter by moving the actuator relative to the shaft to retract the laterally extendable assembly, and removing the apparatus from the medullary cavity.

According to a third aspect of the present invention there is provided an apparatus for forming a canal in a medullary cavity of a bone comprising a shaft having a proximal portion and a distal portion, a laterally extendable assembly operatively coupled to the distal portion of the shaft, the laterally extendable assembly comprises an inflatable element, and an actuator configured for inflating and deflating the inflatable element to adjust an external diameter of the inflatable element.

In a further implementation form of the first, second and/or third aspects, the external diameter is in a range of 4 to 20 millimeters.

In a further implementation form of the first, second and/or third aspects, the laterally extendable assembly is configured to transition between a plurality of discrete steps distributed between the retracted state and the extended state.

In a further implementation form of the first, second and/or third aspects, the laterally extendable assembly is configured to transition continuously between the retracted state and the extended state.

In a further implementation form of the first, second and/or third aspects, the distal end of the shaft is pointed.

In an optional implementation form of the first, second and/or third aspects, the apparatus comprises a locking element configured to limit movement of the actuator with respect to the shaft and prevent change in extension and/or retraction of the laterally extendable assembly.

In an optional implementation form of the first, second and/or third aspects, the apparatus comprises a dial and a pointer mechanically connected to the actuator for indicating the external diameter.

In a further implementation form of the first and/or second aspects, the laterally extendable assembly comprises a plurality of laterally extendable elements disposed along at least part of the shaft and mechanically coupled to the shaft via the actuator. The actuator comprises a joint assembly having a plurality of rods which are tilt-able for retracting and extending the laterally extendable elements.

In a further implementation form of the first and/or second aspects, the joint assembly comprises a first joint assembly and a second joint assembly each comprising a plurality of rods. The first joint assembly is fixed in a longitudinal axis of the shaft and the second joint assembly is moveable in the longitudinal axis of the shaft. A tilt of the plurality of rods is proportional to a distance between the first joint assembly and the second joint assembly. The distance is adjustable by moving the second joint assembly in the longitudinal axis via a knob mounted at a proximal end of the shaft.

In a further implementation form of the first and/or second aspects, the shaft having a threaded exterior goes through a threaded bore of the first joint assembly and a threaded bore of the second joint assembly. The threaded shaft is rotatable via the knob such that rotation of the knob rotates the threaded shaft thus the moveable second joint assembly is moved in the longitudinal axis of the threaded shaft with respect to the fixed first joint assembly and the distance between the first joint assembly and the second joint assembly is adjusted accordingly.

In a further implementation form of the first and/or second aspects, the knob comprises a polygon shaped socket and/or a polygon shaped protrusion at a surface opposite the shaft for attachment of a rotation handle.

In a further implementation form of the first and/or second aspects, the shaft comprises an inner shaft disposed in a bore of an outer hollow shaft such that the inner shaft protrudes out of the outer shaft, the inner shaft is fixed to a fixed section of the knob while the outer shaft mechanically coupled to a second section of the knob is moveable in the longitudinal axis or vice versa. The first joint assembly is disposed on the fixed shaft and the second joint assembly is disposed on the moveable shaft, linear movement of the first section of the knob with respect to the second section of the knob moves the inner shaft with respect to the outer shaft in the longitudinal axis thus the moveable second joint assembly is moved in the longitudinal axis of the shaft with respect to the fixed first joint assembly and the distance between the first joint assembly and the second joint assembly is adjusted accordingly.

In an optional implementation form of the first and/or second aspects, the apparatus comprises one or more spring elements configured to transition between compressed and released states. A tilt of the plurality of rods is proportional to compression of the one or more spring elements.

In an optional implementation form of the first and/or second aspects, the apparatus comprises one or more inflatable elements configured to transition between inflated and deflated states. A tilt of the plurality of rods is proportional to inflation of the one or more inflatable elements.

In an optional implementation form of the first and/or second aspects, one or more of the plurality of laterally extendable elements is shaped to have an outward bent edge.

In an optional implementation form of the first and/or second aspects, one or more of the plurality of laterally extendable elements is shaped to have a sharpened edge.

In an optional implementation form of the first and/or second aspects, the laterally extendable assembly comprises an annular spring element disposed along at least part of the shaft and mechanically coupled to the shaft via the actuator. The actuator comprises a joint assembly comprising a first joint assembly fixed in a longitudinal axis of the shaft and a second joint assembly moveable in the longitudinal axis of the shaft. Extension and retraction of the annular spring element is proportional to a distance between the first joint assembly and the second joint assembly. The distance is adjustable by moving the second joint assembly in the longitudinal axis via a knob mounted at a proximal end of the shaft.

In an optional implementation form of the first and/or second aspects, the laterally extendable assembly comprises a plurality of laterally extendable elements disposed along at least part of the shaft and mechanically coupled to the shaft via the actuator which comprises a conic piston moveable along a longitudinal axis of the shaft for inducing transition of the laterally extendable assembly between a retracted state and an extended state to adjust an external diameter of the laterally extendable assembly.

In an optional implementation form of the first, second and/or third aspects, the medullary canal is formed for insertion of a prosthesis implant.

In an optional implementation form of the first, second and/or third aspects, the apparatus is used to create the guide hole in the medullary cavity.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks automatically. Moreover, according to actual instrumentation and equipment of embodiments of the method and/or system of the invention, several selected tasks could be implemented by hardware, by software or by firmware or by a combination thereof using an operating system.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars are shown by way of example and for purposes of illustrative discussion of embodiments of the invention. In this regard, the description taken with the drawings makes apparent to those skilled in the art how embodiments of the invention may be practiced.

In the drawings:

FIG. 1A, FIG. 1B, FIG. 1C, and FIG. 1D are perspective views, front views, side views, top views, and bottom views of a first exemplary embodiment of an apparatus having an adjustable diameter for forming a canal in bone medullary cavity in retracted and extended states, according to some embodiments of the present invention;

FIG. 2A, FIG. 2B, and FIG. 2C are perspective views, front views, side views, top views, and bottom views of a second exemplary embodiment of an apparatus having an adjustable diameter for forming a canal in bone medullary cavity in retracted and extended states, according to some embodiments of the present invention;

FIG. 3A, FIG. 3B, and FIG. 3C are perspective views, front views, side views, top views, and bottom views of a third exemplary embodiment of an apparatus having an adjustable diameter for forming a canal in bone medullary cavity in retracted and extended states, according to some embodiments of the present invention;

FIG. 4A, FIG. 4B, and FIG. 4C are perspective views, front views, side views, top views, and bottom views of a fourth exemplary embodiment of an apparatus having an adjustable diameter for forming a canal in bone medullary cavity in retracted and extended states, according to some embodiments of the present invention;

FIG. 5A and FIG. 5B are perspective views, front views, side views, top views, and bottom views of a fifth exemplary embodiment of an apparatus having an adjustable diameter for forming a canal in bone medullary cavity in retracted and extended states, according to some embodiments of the present invention;

FIG. 6 presents perspective and side views of an exemplary handle attached to operate an apparatus having an adjustable diameter for forming a canal in bone medullary cavity in retracted and extended states, according to some embodiments of the present invention;

FIG. 7A, FIG. 7B, and FIG. 7C are perspective views, front views, side views, top views, and bottom views of a sixth exemplary embodiment of an apparatus having an adjustable diameter for forming a canal in bone medullary cavity in retracted and extended states, according to some embodiments of the present invention;

FIG. 8 presents perspective and side views of an exemplary apparatus having an adjustable diameter for forming a canal in bone medullary cavity with various end tips, according to some embodiments of the present invention; and

FIG. 9 is a flowchart of an exemplary process for using apparatus having an adjustable diameter to form a canal in a bone medullary cavity, according to some embodiments of the present invention.

### DETAILED DESCRIPTION OF EMBODIMENTS OF THE INVENTION

The present invention, in some embodiments thereof, relates to an apparatus for forming a canal in bone medullary cavity and, more particularly, but not exclusively, to an apparatus having an adjustable diameter for forming a canal in bone medullary cavity.

According to some embodiments of the present invention there is provided an apparatus and methods for use thereof to extract bone marrow and form a canal (interchangeably designated medullary canal herein after) in the medullary cavity of one or more bones and extract bone marrow from the cavity during and/or in preparation for one or more medical and/or surgical procedures. For example, in preparation for a shoulder joint replacement, for example, reverse shoulder, total shoulder, and/or the like the apparatus may be used to form a medullary canal in the humerus bone in which a prosthesis is to be implanted.

In particular, the apparatus may be configured and operated to dynamically adjust a diameter of a section (portion) of the apparatus which is inserted into the medullary cavity such that the apparatus may be used as a single tool to form the medullary canal having a desired diameter and potentially eliminate the need for multiple different tools having different diameters.

The apparatus may comprise a shaft having a proximal portion and a distal portion inserted into the medullary cavity to form the medullary canal. The apparatus may further include an actuator, for example, a mechanical actuator, configured to be moveable relative (with respect) to the shaft and a laterally extendable assembly operatively coupled to the distal portion of the shaft which is configured to transition between a retracted (closed) state and an extended (open) state in a lateral axis perpendicular to a longitudinal of the shaft.

In particular, movement of the actuator relative to the shaft, for example, rotational movement, linear movement, lateral movement, and/or the like may induce transition of the laterally extendable assembly between the retracted and extended states in a lateral axis perpendicular to a longitudinal axis of the shaft thus adjusting a diameter, specifically an external diameter, of the laterally extendable assembly.

Operating the apparatus to form a canal in the bone medullary cavity of a bone may include inserting the distal (leading) portion of the apparatus into a guide hole formed in the medullary cavity, optionally using the apparatus. Since the laterally extendable assembly is mechanically coupled to the distal portion of the shaft which is the portion inserted into the medullary cavity to form the canal, the external diameter of the laterally extendable assembly may define the diameter of the medullary canal formed in the bone.

The external diameter of the laterally extendable assembly, which is defined by the level of extension of the laterally extendable assembly in the lateral axis, may be adjusted in a range of, for example, 4-20 millimeters (mm) such that when fully retracted, the external diameter of the laterally extendable assembly may be 4 mm and when fully extended, the external diameter of the laterally extendable assembly may be 20 mm. Obviously, the apparatus may be configured, designed, and/or constructed to facilitate other ranges of the external diameter, for example, 5-16 mm, 4-16 mm, 5-20 mm, and/or the like.

According to some embodiments, the laterally extendable assembly may be configured to transition between a plurality of discrete steps distributed between the retracted state and the extended state where each step corresponds to a respective external diameter of the laterally extendable assembly. Continuing the previous example, assuming the external diameter range is configured to 4-20 mm, the laterally extendable assembly may be configured to transition between 17 steps each corresponding to a respective external diameter, for example, 4 mm, 5 mm, 6 mm, 7 mm, 8 mm, and so on to 20 mm.

Optionally, the laterally extendable assembly may be configured to transition continuously between the retracted state and the extended state and is thus adjustable to have practically any external diameter within the extension range. For example, assuming the external diameter range is configured to 4-20 mm, the laterally extendable assembly may be configured to have any external diameter within that range.

The distal end of the shaft may be configured to have a pointed tip which may serve to improve penetration of the shaft into the medullary cavity.

The actuator may be configured to move and extend and/or retract the laterally extendable assembly while the apparatus, specifically its portion comprising the laterally extendable assembly is inside the medullary cavity thus eliminating the need to remove the apparatus from the medullary canal in order to change its diameter.

Optionally, the apparatus may further include a dial and a pointer mechanically connected to the actuator for indicating the external diameter. This means that the dial and pointer may be configured to express the external diameter of the laterally extendable assembly which is derived from the movement of the actuator relative to the shaft.

Optionally, the apparatus comprises one or more locking elements configured to lock the actuator and limit its movement with respect to the shaft in order to prevent movement of the laterally extendable assembly, i.e., to prevent the laterally extendable assembly from retracting and/or extending. The locking element may therefore ensure that the external diameter of the laterally extendable assembly does not change while the locking element is locked. The locking element may be released to move the actuator with respect to the shaft in order to expand and/or retract the laterally extendable assembly and adjust its diameter accordingly.

Optionally, the laterally extendable element and/or part thereof may be configured to have an outward bent edge. Optionally, one or more of the laterally extendable elements may be shaped to comprise one or more slots (e.g., slit, flute, groove, aperture, etc.) and/or the like. Optionally, one or more edges of the laterally extendable element may be shaped to have a sharpened edge to form a razor like edge.

The adjustable diameter apparatus for forming a canal in bone medullary cavity may present major benefits and advantages over currently existing methods and tools for creating and forming canals in bone medullary cavity.

It is a particular feature of present invention that since the diameter of the adjustable diameter apparatus, specifically the diameter of the apparatus's portion which is inserted into the bone medullary cavity, may be dynamically adjusted, it may be used as a single tool, translating into reduction of cost, procedure complexity and maintenance compared to currently available methods which may use multiple different tools having different diameters.

It is a particular feature of present invention that the actuator of the adjustable diameter apparatus is configured to move with respect to the shaft while the shaft of the apparatus is inserted in the bone medullary cavity thus eliminating the need to remove the apparatus in order to change its diameter as may be done by the existing methods in which each tool having a fixed diameter needs to be removed and replaced with another tool having a larger diameter. Complexity and/or time (duration) of the medullary canal forming procedure may be therefore significantly reduced compared to the existing methods. In addition, using a single apparatus and thus reducing the number of extractions and insertions of tools into the medullary cavity may reduce the risk of damaging the medullary cavity and/or other bone, muscle tissue and/or other organs of the patient compared to the existing methods in which tools are inserted and extracted in and out of the medullary cavity multiple times.

It is a particular feature of present invention that, the adjustable diameter apparatus may be optionally used to create an initial guide hole in the bone for inserting the shaft of the apparatus into the bone medullary cavity. This may further reduce the number of tools required for the procedure since the use of the adjustable diameter apparatus may eliminate the need for a specific tool for creating the guide hole as may be done by the existing methods.

It is a particular feature of present invention that the bent and/or razor like shaped edge(s) of the laterally extendable element may significantly improve its capability and/or capacity to extract bone marrow in the medullary cavity and effectively form the medullary canal.

It is a particular feature of present invention that the laterally extendable element may be configured to bend outwards, comprise one or more slots and/or flutes and/or shaped with sharp edges configured to further improve its capability and/or capacity to extract bone marrow in the medullary cavity and effectively form the medullary canal.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

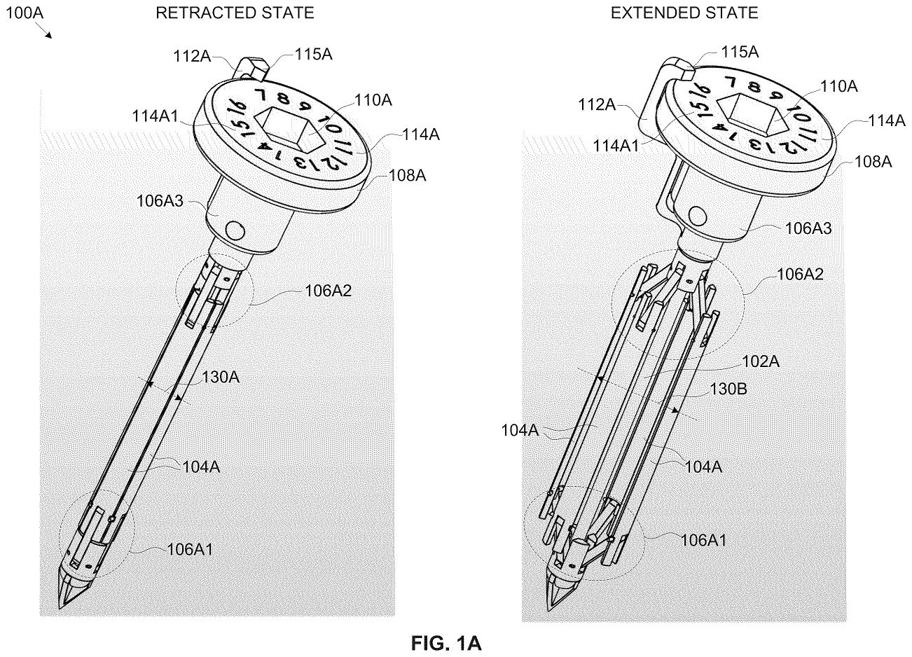

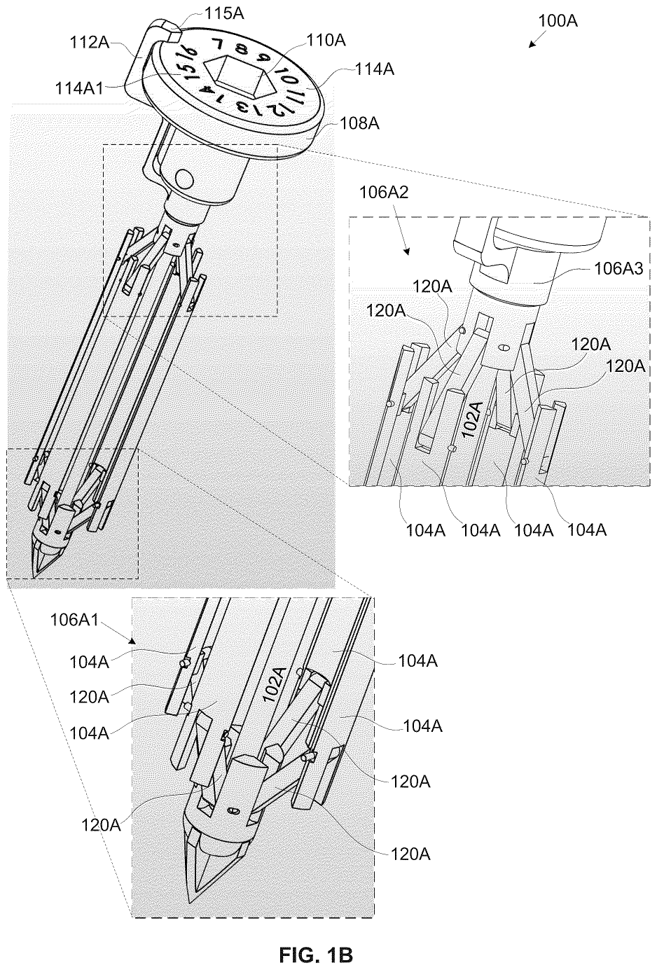

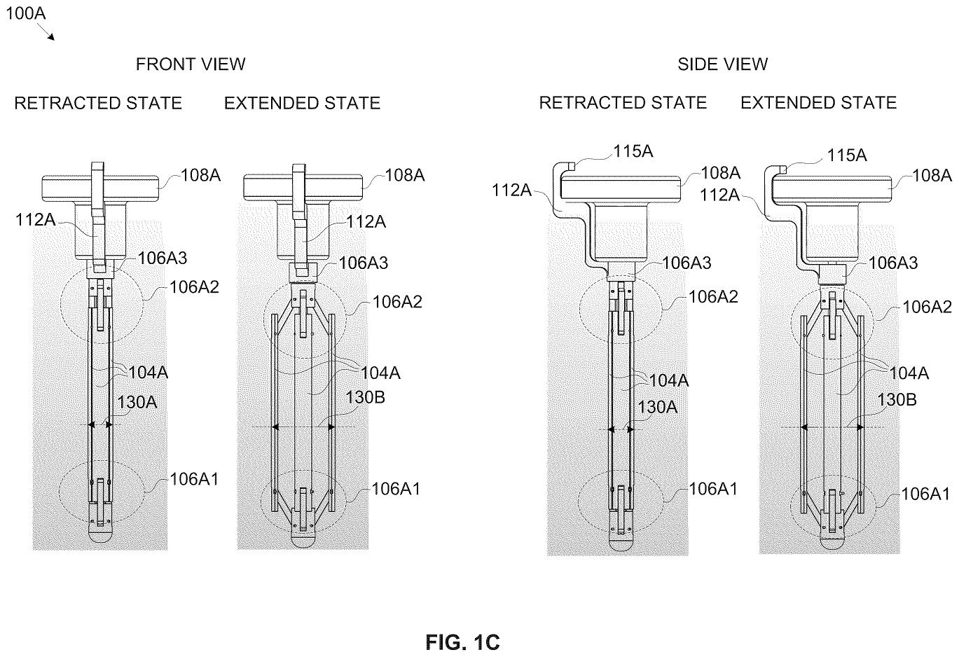

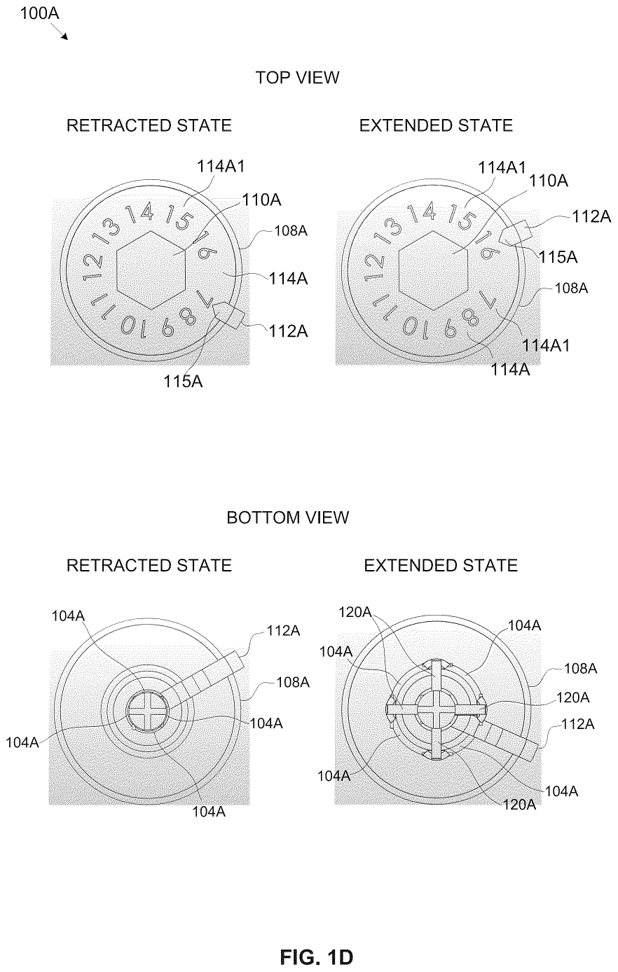

Referring now to the drawings, FIG. 1A, FIG. 1B, FIG. 1C, and FIG. 1D are perspective views, front views, side views, top views, and bottom views of a first exemplary embodiment of an apparatus having an adjustable diameter for forming a canal in bone medullary cavity in retracted and extended states, according to some embodiments of the present invention.

An exemplary apparatus 100A configured and operated to form a canal in the medullary cavity of one or more bones may comprise a shaft 102A, an actuator 106A and a laterally extendable assembly comprising a plurality of laterally extendable elements 104A, for example, four elements operatively coupled to the shaft 102A, specifically to a distal portion of the shaft 102A which is configured for insertion into the bone medullary cavity during the canal forming procedure.

In particular, the plurality of laterally extendable elements 104A may be disposed along at least part of the shaft 102A, for example, at a distal portion of the shaft and mechanically coupled to the shaft 102A via the actuator 106A which may comprise a joint assembly, specifically a first joint assembly 106A1 and a second joint assembly 106A2. Each of the joint assemblies 106A1 and 106A2 may comprise a plurality of rods 120A which are tilt-able for retracting and extending the laterally extendable elements 104A.

The actuator 106A may be configured such that it is moveable relative to the shaft 102 by configuring the first joint assembly 106A1 to be fixed (fixed) in a longitudinal axis of the shaft 102A while configuring the second joint assembly 106A2 to be moveable in the longitudinal axis of the shaft 102A.

As such when the second joint assembly 106A2 is moved, the distance between the first joint assembly 106A1 and the second joint assembly 106A2 is changed, for example, when the second joint assembly 106A2 is moved towards the first joint assembly 106A1, the distance is reduced and when the second joint assembly 106A2 is moved away from the first joint assembly 106A1, the distance is increased.

The plurality of tilt-able rods 120A connect the laterally extendable elements 104A to the shaft 102A via hinges such that the rods 120A may tilt and thus extend or retract the laterally extendable elements 104A. Evidently, since the rods 120A are connected to the fixed first joint assembly 106A1 and to the moveable second joint assembly 106A2 on one side and to the laterally extendable elements 104A on the other side, the tilt of the plurality of rods 120A is proportional to the distance between the first joint assembly 106A1 and the second joint assembly 106A2.

Movement of the actuator 106A, specifically movement of the second joint assembly 106A2 may therefore induce transition of the laterally extendable elements 104A between retracted and extended states. Extension and retraction of the laterally extendable elements 104A may define a diameter of the laterally extendable elements 104A, in particular an external diameter 130 between the edges of opposite laterally extendable elements 104A. A seen, in the fully retracted state, the external diameter 130 between the edges of opposite laterally extendable elements 104A may be a minimum external diameter 130A while in the fully extended state, the external diameter 130 between the edges of opposite laterally extendable elements 104A may be a maximal external diameter 130B.

Movement of the second joint assembly 106A2 may be controlled to set the laterally extendable elements 104A in a plurality of extension states between the fully retracted state and the fully extended state which may define the external diameter 130 accordingly in a range between the minimal external diameter 130A and the maximal external diameter 130B. The apparatus 100A may be configured such that the external diameter 130 may be in one or more ranges. For example, the apparatus 100A may be configured such that the external diameter 130 in a range of 4-20 mm, such that the minimal external diameter 130A is 4 mm and the maximal external diameter 130B is 20 mm. In another example, the apparatus 100A may be configured such that the external diameter 130 may be in a range of 7-16 mm, such that the minimal external diameter 130A is 7 mm and the maximal external diameter 130B is 16 mm.

One or more methods may be applied to access and/or operate the actuator 106A to move the moveable second joint assembly 106A2 in the longitudinal axis of the shaft 102A and thus change the distance between the first joint assembly 106A1 and the second joint assembly 106A2. For example, the actuator 106A may be operated via a knob 108A mounted on the proximal end of the shaft 102A.

For example, the shaft 102A may be configured to have a threaded exterior which goes through a threaded bore of the first joint assembly 106A1 and through a threaded bore of the second joint assembly 106A2. The threaded shaft 106A is rotatable via the knob 108A such that rotation of the knob 108A rotates the threaded shaft 106A. As result of rotation of the threaded shaft 102A, the moveable second joint assembly 106A2 may be moved in the longitudinal axis of the threaded shaft 102A with respect to the fixed first joint assembly 106A1 and the distance between them is adjusted accordingly. For example, rotating the knob 108A in one direction, for example, clockwise may cause the threaded shaft 106A to rotate clockwise and pull the second joint assembly 106A2 which may include, for example, a floating nut 106A3 such that the second joint assembly 106A2 is moved towards the first joint assembly 106A1 which is fixed in place. In another example, rotating the knob 108A in the other direction, for example, counter-clockwise may cause the threaded shaft 106A to rotate counter-clockwise and push the second joint assembly 106A2 such that the second joint assembly 106A2 is moved away from the fixed first joint assembly 106A1.

In such embodiments the knob 108A may comprise a polygon shaped socket (groove) 110A and/or a polygon shaped protrusion at a surface opposite the shaft 102A for attachment of a handle (power tool), manual and/or electrically or hydraulically powered, for example, a wrench, a ratchet, an Allen key, a hex key, a Torx key, and/or the like configured to rotate the knob 108A. The polygon shape socket 110A and/or protrusion may take one or more forms and/or structures, for example, a triangle, a square, hexagon, a Torx, and/or the like.

In another example, a shaft such as the shaft 102A may comprise an inner shaft disposed in a bore of an outer hollow shaft such that the inner shaft protrudes out of the outer shaft. The inner shaft may be fixed to a first (fixed) section of a knob 108A while the outer shaft mechanically may be coupled to a second section of a knob such as the knob 108A separate from the first section such that the outer shaft may be moveable relative to the inner shaft, specifically in the longitudinal axis of the shaft 102. In such case, the fixed first joint assembly 106A1 may be mechanically coupled to the inner shaft of the shaft 102A while the moveable second joint assembly 106A2 may be mechanically coupled to the outer shaft of the shaft 102A.

The knob 108A may be configured to convert linear movement, force, and/or pressure on its top surface, opposite the shaft 102A, into movement of the outer shaft relative to the inner shaft. As such, applying linear movement, force, and/or pressure in one direction, for example, downwards, may cause the second joint assembly 106A2 which is coupled to the moving outer shaft to move towards the first joint assembly 106A1 coupled to the static inner shaft. Complementary, applying linear movement, force, and/or pressure on the knob 108A in the other direction, for example, upwards, may cause the second joint assembly 106A2 to move away from the first joint assembly 106A1.

Alternatively, the inner shaft may be moveable while the outer shaft may be fixed. In such case, the same design may apply with the exception that the fixed first joint assembly 106A1 may be operatively coupled to the outer shaft of the shaft 102A while the moveable second joint assembly 106A2 may be mechanically coupled to the inner shaft of the shaft 102A.

The apparatus 100A may further include one or more locking 112A elements configured to lock the actuator 106A, specifically the second joint assembly 106A2 and limit its movement with respect to the shaft 102A in order to prevent movement of the laterally extendable elements 104A, i.e., to prevent the laterally extendable assembly from retracting and/or extending. The locking element may be released to move the second joint assembly 106A2 with respect to the shaft 102A in order to expand and/or retract the laterally extendable elements 104A and adjust the external diameter 130 accordingly.

As such, while the locking element(s) 112A is in its released state, the apparatus 100A may be operated to move the actuator 106A, specifically the second joint assembly 106A2 to a certain position relative to the shaft 102A to set a desired external diameter 130 of the laterally extendable elements 104A. Once the second joint assembly 106A2 is in the certain position, the locking element(s) 112A may be switched to their lock state in order to ensure that the second joint assembly 106A2 does not move such that the laterally extendable elements 104A are locked in their place and their external diameter 130 does not change.

The locking element(s) 112A may employ one or more configurations, and/or designs to enable locking and releasing movement of the actuator 106A, specifically movement of the second joint assembly 106A2. For example, the locking element(s) 112A may be connected to the shaft 102A via one or more hinges such that it may move in a lateral axis with respect to the shaft 102A and to the second joint assembly 106A2. The locking element(s) 112A may be shaped to have a pointed distal tip shaped to fit into each of a plurality of depressions and/or slits embedded in the second joint assembly 106A2. As such, pushing the proximal end of the locking element(s) 112A outwards, i.e., away from the knob 108A, may cause the distal pointed tip of the locking element(s) 112A to enter into one of the slits of the second joint assembly 106A2 and prevent its rotation and/or liner movement thus preventing its movement relative to the shaft 102A.

Taking advantage of the locking element(s) 112A, in addition to using the knob 108A for operating the actuator 106A, specifically move the second joint assembly 106A2 to extract or retract the laterally extendable elements 104A and adjust their external diameter 130 accordingly, the knob 108A may be further used to rotate the shaft 102A and the laterally extendable elements 104A, for example, while the shaft 102A and the laterally extendable elements 104A are inserted in the medullary cavity for forming the canal.

In such embodiments, while the locking element(s) 112A is released, rotating the knob 108A may extract or retract the laterally extendable elements 104A and adjust their external diameter 130 accordingly. However, while the locking element(s) 112A is locked, rotating the knob 108A may rotate the apparatus 100A, specifically the shaft 102A and the laterally extendable elements 104A.

Optionally, the apparatus 100A may comprise a dial 114A and a pointer 115A indicative of the external diameter 130 of the laterally extendable elements 104A. The dial 114A may comprise one or more marks, digits and/or symbols indicative of the external diameter 130. For example, the dial 114A may comprise a plurality of digits expressing a range of the external diameter 130 in mm, for example, 7 mm to 16 mm. The pointer 115A may point at one of the digits which corresponds to the current external diameter 130 of the laterally extendable elements 104A. In order to illustrate the symbols, in this case digits of the dial 114A, the digit 15 which corresponds, for example, to an external diameter 130 of 15 mm is marked with numeral 114A1. However, to avoid clutter and maintain readability of the drawings, the other symbols (digits) of the dial 114A are not associated with numerals.

The dial 114A and the pointer 115A may be implemented using one or more techniques and/or designs. For example, since the external diameter 130 of the laterally extendable elements 104A may be derived from the position of the actuator 106A, specifically the position of the second joint assembly 106A2 in the longitudinal axis of the shaft 102A, the pointer 115A may be mechanically coupled to the second joint assembly 106A2 such that it may express movement of the second joint assembly 106A2. As such, the position of the pointer 115A with respect to the dial 114A may change according to the movement of the second joint assembly 106A2 and point at the mark, for example, the digit corresponding to the external diameter 130 of the laterally extendable elements 104A defined by the position of the second joint assembly 106A2.

Optionally, the pointer 115A may be integrated in one or more of the locking elements 112A which may be also coupled to the second joint assembly 106A2 to limit its movement relative to the shaft 102A in the longitudinal axis of the shaft 102A.

It should be noted that while in the description of the apparatus 100A the first joint assembly 106A1 is fixed while the second joint assembly 106A2 is moveable, this should not be construed as limiting, since as may become apparent to a person skilled in the art the roles of the first and second joint assemblies 106A1 and 106A2 may be switched such that the second joint assembly 106A2 is fixed while the first joint assembly 106A1 is moveable. Moreover, other structures may be designed in which both the first and second joint assemblies 106A1 and 106A2 may be moveable with respect to each other.

Optionally, one or more of the laterally extendable elements 104A may be shaped to have an outward bent edge, for example, a L-shaped edge, and/or the like to improve extraction of bone marrow and formation of the medullary canal when the laterally extendable elements 104A are rotated in the medullary cavity.

Optionally, one or more of the laterally extendable elements 104A may be shaped to have a sharpened edge to further improve extraction of bone marrow and formation of the medullary canal when the laterally extendable elements 104A are rotated in the medullary cavity.

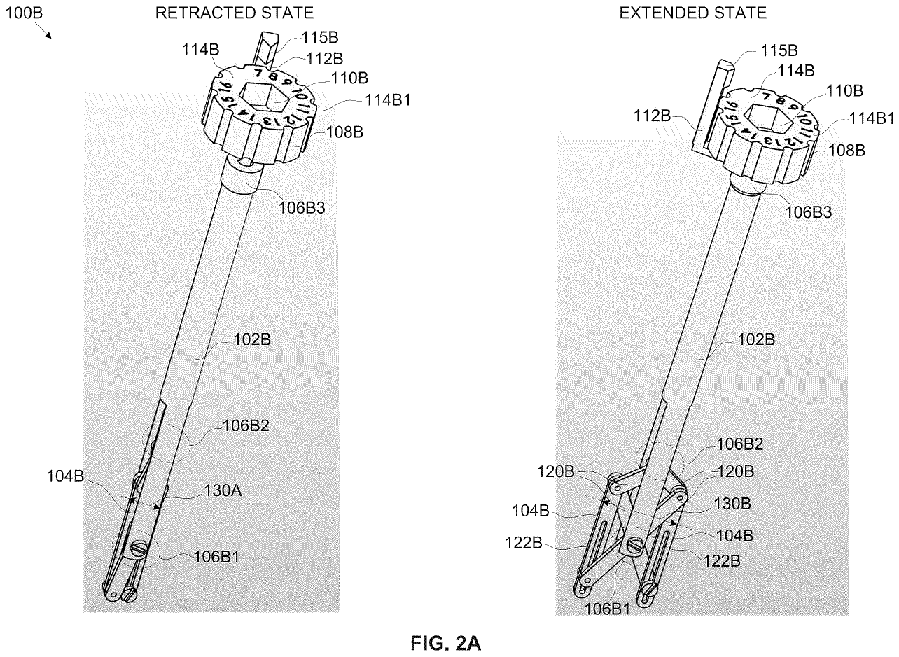

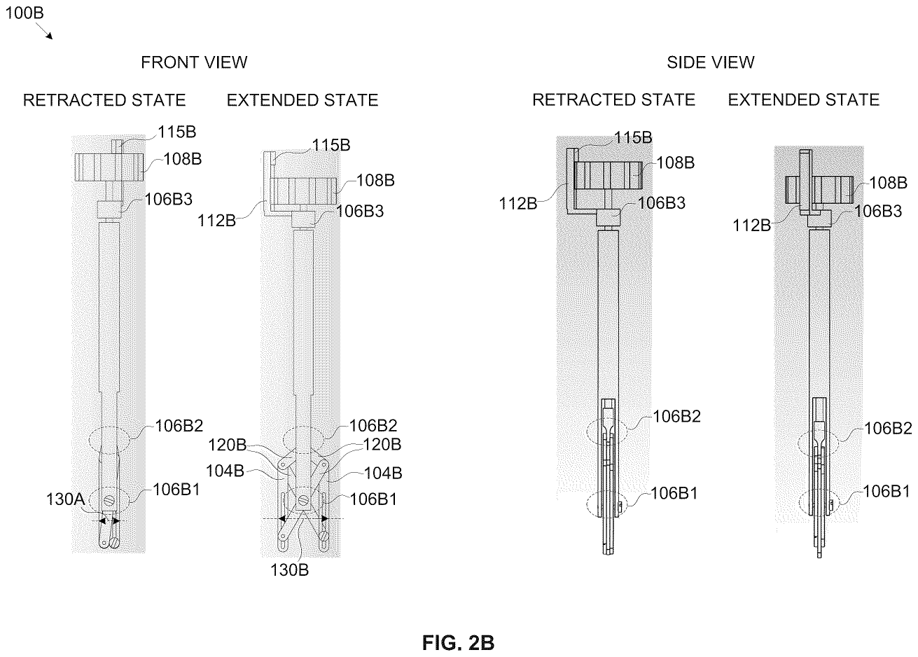

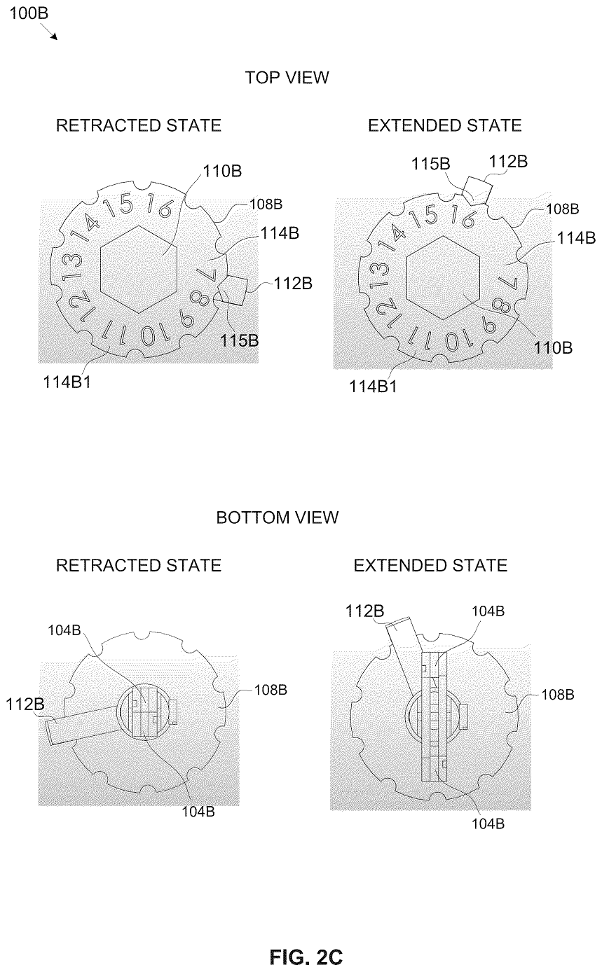

Reference is now made to FIG. 2A, FIG. 2B, and FIG. 2C, which are perspective views, front views, side views, top views, and bottom views of a second exemplary embodiment of an apparatus having an adjustable diameter for forming a canal in bone medullary cavity in retracted and extended states, according to some embodiments of the present invention.

An exemplary apparatus 100B configured and operated to form a canal in the medullary cavity of one or more bones may comprise a shaft 102B, an actuator 106B and a laterally extendable assembly comprising a plurality of laterally extendable elements 104B, for example, two elements operatively coupled to the shaft 102B, specifically to a distal portion of the shaft 102B which is configured for insertion into the bone medullary cavity during the canal forming procedure.

The construction of the apparatus 100B, its operational and structural elements are very similar to those of the apparatus 100A described herein before.

In particular, similarly to the actuator 106A, the actuator 106B may comprise a joint assembly, specifically a first joint assembly 106B1 which is fixed relative to the shaft 102B in the longitudinal axis of the shaft 102B and a second joint assembly 106B2 moveable in the longitudinal axis. Each of the joint assemblies 106B1 and 106B2 may comprise a plurality of rods 120B which are tilt-able for retracting and extending the laterally extendable elements 104B.

As described for the apparatus 100A, extension and retraction of the laterally extendable elements 104B, which is defined by the level of tilting of the rods 120B which in turn depends on the distance between the moveable second joint assembly 106B2 and the fixed first joint assembly 106B1, may define the external diameter 130 of the laterally extendable elements 104B, i.e., the distance between the edges of the opposite laterally extendable elements 104B.

As described for the apparatus 100A, movement of the actuator 106B, specifically movement of the second joint assembly 106B2 may induce transition of the laterally extendable elements 104B between (and including) a fully retracted state and a fully extended state, and hence define the external diameter 130 between the edges of opposite laterally extendable elements 104B accordingly in a range between a minimal external diameter 130A in the fully retracted state and a maximal external diameter 130B in the fully extended state.

Similarly to the apparatus 100A, movement of the second joint assembly 106B2 to set its distance from the first joint assembly 106B1 may be controlled via rotation and/or linear movement of a knob 108B similarly to the knob 108A. for example, in case of a threaded shaft 102A, the movement of the second joint assembly 106B2 may be controlled via a floating nut 106B3 similarly to the floating nut 106A3. Moreover, in such embodiments the knob 108B may comprise a polygon shaped socket (groove) 110B and/or a polygon shaped protrusion at a surface opposite the shaft 102B for attachment of a manual and/or electrically handle (power tool) configured to rotate the knob 108B.

Optionally, the apparatus 100B may comprise one or more locking elements 112B such as the locking elements 112A for locking the position of the moveable second joint assembly 106B2 to maintain the currently set external diameter 130 of the laterally extendable elements 104B.

Optionally, the apparatus 100B may comprise a dial 114B such as the dial 114A and a pointer 115B indicative of the external diameter 130 of the laterally extendable elements 104B. In order to illustrate the symbols, in this case digits of the dial 114B, the digit 11 which corresponds, for example, to an external diameter 130 of 11 mm is marked with numeral 114B1. However, to avoid clutter and maintain readability of the drawings, the other symbols (digits) of the dial 114B are not associated with numerals. The apparatus 100B may employ one or more structures, configurations and/or designs for implementing the dial 114B and the pointer 115B similarly to those used for the dial 104A and its pointer 115B. For example, the pointer 115B may be integrated with one or more of the locking elements 112B.

As stated for the apparatus 100A, while in the description of the apparatus 100B the first joint assembly 106B1 is fixed while the second joint assembly 106B2 is moveable, this should not be construed as limiting, since as may become apparent to a person skilled in the art the roles of the first and second joint assemblies 106B1 and 106B2 may be switched such that the second joint assembly 106B2 is fixed while the first joint assembly 106B1 is moveable. Moreover, other structures may be designed in which both the first and second joint assemblies 106B1 and 106B2 may be moveable with respect to each other.

Optionally, one or more of the laterally extendable elements 104B may be shaped to have an outward bent edge, for example, a L shaped edge, and/or the like to improve extraction of bone marrow and formation of the medullary canal when the laterally extendable elements 104B are rotated in the medullary cavity.

Optionally, one or more of the laterally extendable elements 104B may be shaped to have a sharpened edge to further improve extraction of bone marrow and formation of the medullary canal when the laterally extendable elements 104B are rotated in the medullary cavity. For example, one or more of the laterally extendable elements 104B may be configured and shaped to include one or more elongated slots 122B, for example, a slit, a depression, a groove, an aperture, a flute and/or the like. As such, when the laterally extendable elements 104B are rotated in the medullary cavity, the slots 122B may efficiently and smoothly cut through the bone marrow thus improving its extraction to form the medullary canal.

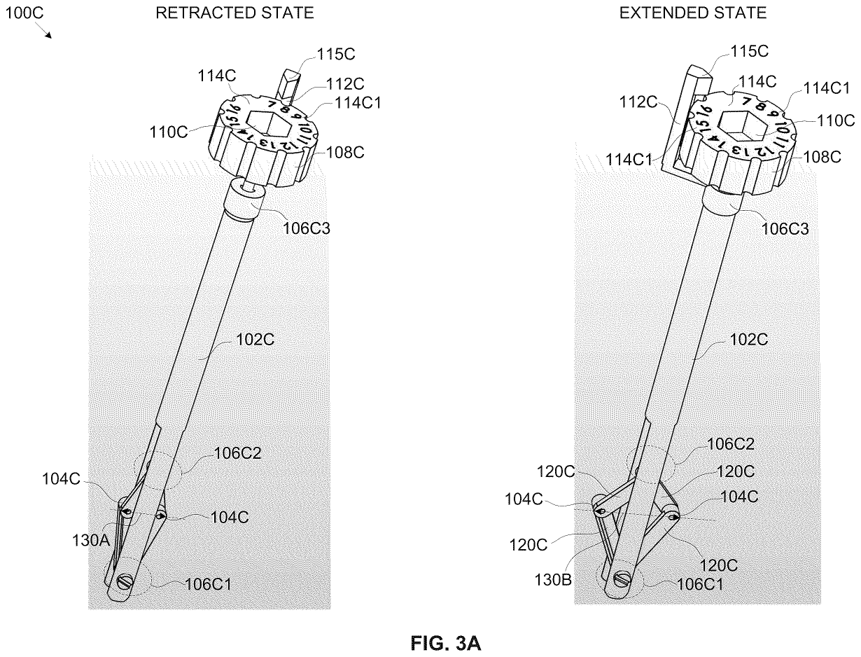

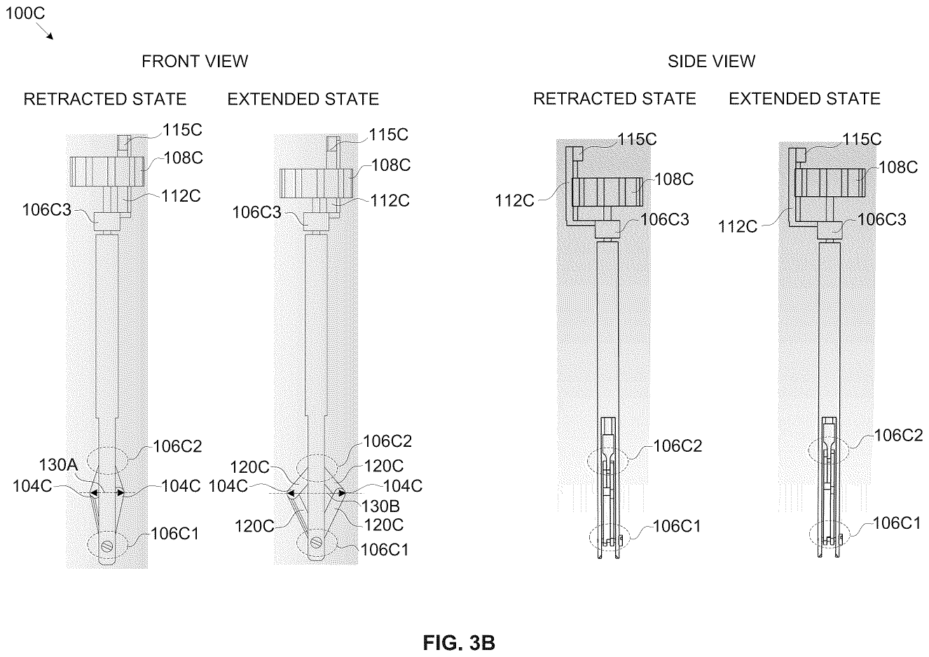

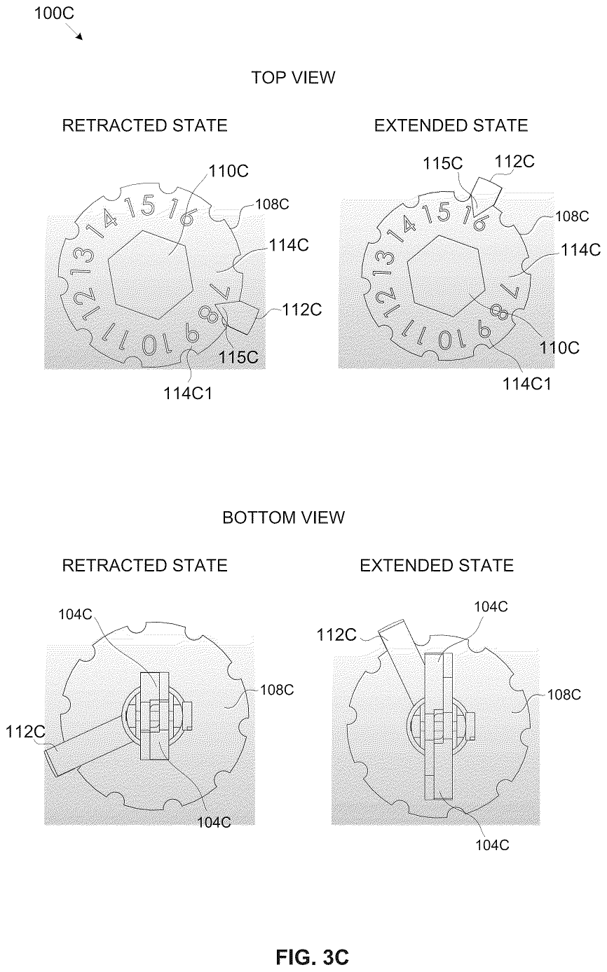

Reference is now made to FIG. 3A, FIG. 3B, and FIG. 3C, which are perspective views, front vies, side views, top views, and bottom views of a third exemplary embodiment of an apparatus having an adjustable diameter for forming a canal in bone medullary cavity in retracted and extended states, according to some embodiments of the present invention.

An exemplary apparatus 100C configured and operated to form a canal in the medullary cavity of one or more bones may comprise a shaft 102C, an actuator 106C and a laterally extendable assembly comprising a plurality of laterally extendable elements 104C, for example, two elements operatively coupled to the shaft 102C, specifically to a distal portion of the shaft 102C which is configured for insertion into the bone medullary cavity during the canal forming procedure.

The construction of the apparatus 100C, its operational and structural elements are very similar to the those of the apparatuses 100A and 100B described herein before.

In particular, similarly to the actuator 106A, the actuator 106C may comprise a joint assembly, specifically a first joint assembly 106C1 which is fixed relative to the shaft 102B in the longitudinal axis of the shaft 102C and a second joint assembly 106C2 moveable in the longitudinal axis. In this embodiment, the laterally extendable elements 104C are formed by a plurality of rods 120C which are tilt-able and thus retract and extend the laterally extendable elements 104C.

As described for the apparatus 100A, extension and retraction of the laterally extendable elements 104C, which is defined the level of tilting of the rods 120C, i.e., of the laterally extendable elements 104C which in turn depends on the distance between the moveable second joint assembly 106C2 and the fixed first joint assembly 106C1, may define the external diameter 130 of the laterally extendable elements 104C, i.e., the distance between the edges of the opposite laterally extendable elements 104C.

As described for the apparatus 100A, movement of the actuator 106C, specifically movement of the second joint assembly 106C2 may induce transition of the laterally extendable elements 104C between (and including) a fully retracted state and a fully extended state, and hence define the external diameter 130 between the edges of opposite laterally extendable elements 104B accordingly in a range between a minimal external diameter 130A in the fully retracted state and a maximal external diameter 130B in the fully extended state.

Similarly to the apparatus 100A, movement of the second joint assembly 106C2 to set its distance from the first joint assembly 106C1 may be controlled via rotation and/or linear movement of a knob 108C such as the knob 108A. for example, in case of a threaded shaft 102C, the movement of the second joint assembly 106C2 may be controlled via a floating nut 106C3 such as the floating nut 106A3. Moreover, in such embodiments the knob 108B may comprise a polygon shaped socket (groove) 110C and/or a polygon shaped protrusion at a surface opposite the shaft 102C for attachment of a manual and/or electrically handle (power tool) configured to rotate the knob 108C.

Optionally, the apparatus 100C may comprise one or more locking elements 112C such as the locking elements 112A for locking the position of the moveable second joint assembly 106C2 to maintain the currently set external diameter 130 of the laterally extendable elements 104C.

Optionally, the apparatus 100C may comprise a dial 114C such as the dial 114A and a pointer 115C indicative of the external diameter 130 of the laterally extendable elements 104C. In order to illustrate the symbols, in this case digits of the dial 114C, the digit 9 which corresponds, for example, to an external diameter 130 of 9 mm is marked with numeral 114C1. However, to avoid clutter and maintain readability of the drawings, the other symbols (digits) of the dial 114C are not associated with numerals. The apparatus 100C may employ one or more structures, configurations and/or designs for implementing the dial 114C and the pointer 115C similarly to those used for the dial 104A and its pointer 115C. For example, the pointer 115C may be integrated with one or more of the locking elements 112C.

As stated for the apparatus 100C, while in the description of the apparatus 100C the first joint assembly 106C1 is fixed while the second joint assembly 106C2 is moveable, this should not be construed as limiting, since as may become apparent to a person skilled in the art the roles of the first and second joint assemblies 106C1 and 106C2 may be switched such that the second joint assembly 106C2 is fixed while the first joint assembly 106C1 is moveable. Moreover, other structures may be designed in which both the first and second joint assemblies 106C1 and 106C2 may be moveable with respect to each other.

Optionally, one or more of the laterally extendable elements 104C may be shaped to have an outward bent edge, for example, a L shaped edge, and/or the like to improve extraction of bone marrow and formation of the medullary canal when the laterally extendable elements 104C are rotated in the medullary cavity.

Optionally, one or more of the laterally extendable elements 104C may be shaped to have a sharpened edge to further improve extraction of bone marrow and formation of the medullary canal when the laterally extendable elements 104C are rotated in the medullary cavity.

In another example, an apparatus such as the apparatuses 100A, 100B, and/or 100C, collectively designated apparatus 100 may employ one or more spring elements configured to transition between compressed and released states. The tilt of a plurality of rods such as the rods 120A, 120B, and/or 120C is proportional to compression of the spring element(s).

For example, an apparatus 100 constructed with a plurality of laterally extendable elements such as the laterally extendable elements 104A of the apparatus 100A, may comprise an actuator 106 comprising one or more spring elements disposed at one or more locations along the distal portion of a shaft such as the shaft 102A, for example, beneath a first joint element such as the first joint assembly 106A1 and beneath a second joint assembly such as the second joint assembly 106A2. The spring elements may be operated to transition between their compressed and released states via a knob such as the knob 108A which may be operatively coupled to the spring elements, for example, via a rotatable metal cord configured to compress or retract the spring elements when rotated.

In another example, an apparatus such as the apparatuses 100A, 100B, and/or 100C, collectively designated apparatus 100 may employ one or more inflatable elements configured to transition between inflated and deflated states. The tilt of a plurality of rods such as the rods 120A, 120B, and/or 120C is proportional to inflation of the spring element(s).

For example, an apparatus 100 constructed with a plurality of laterally extendable elements such as the laterally extendable elements 104B of the apparatus 100B, may comprise an actuator 106 comprising one or more inflatable elements disposed at one or more locations along the distal portion of a shaft such as the shaft 102B, for example, beneath a first joint element such as the first joint assembly 106B1 and beneath a second joint assembly such as the second joint assembly 106B2. The inflatable elements may be operated to transition between their inflated and deflated states via a knob such as the knob 108B which may be operatively coupled to the spring elements, for example, via a pressure tube configured to pump fluid, for example, liquid, gas (e.g. air), and/or the like into the inflatable elements or release air from the inflatable elements thus inflating or deflating the inflatable elements respectively. Moreover, the apparatus 100 may further include a pump, for example, a manual pump, an electric pump, and/or the like configured to push fluid in or release fluid from the inflatable element.

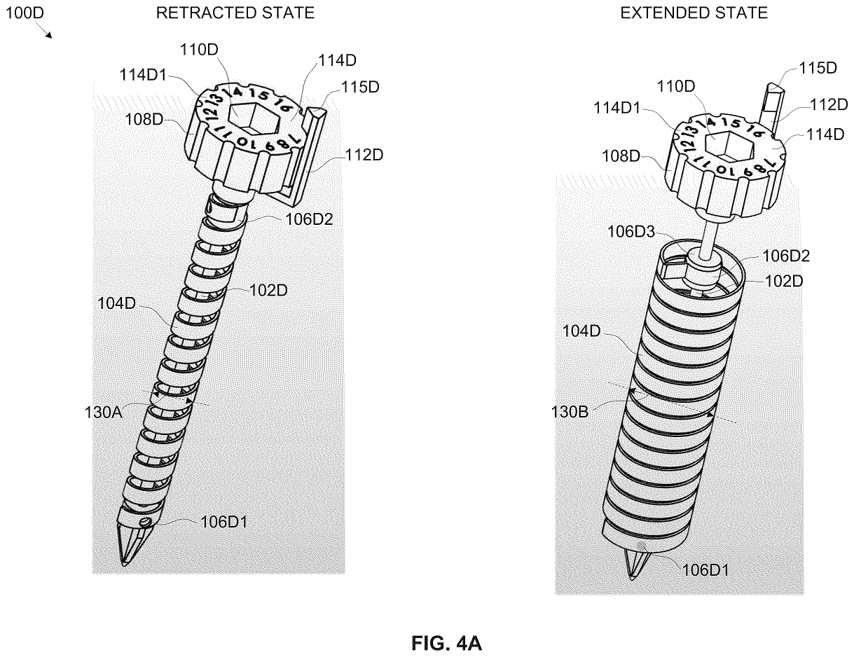

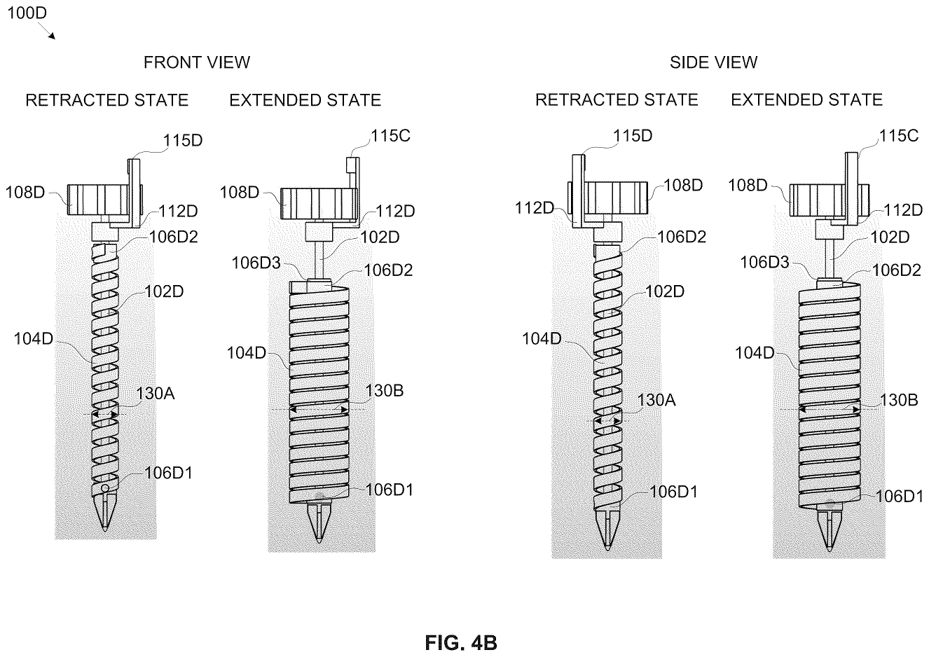

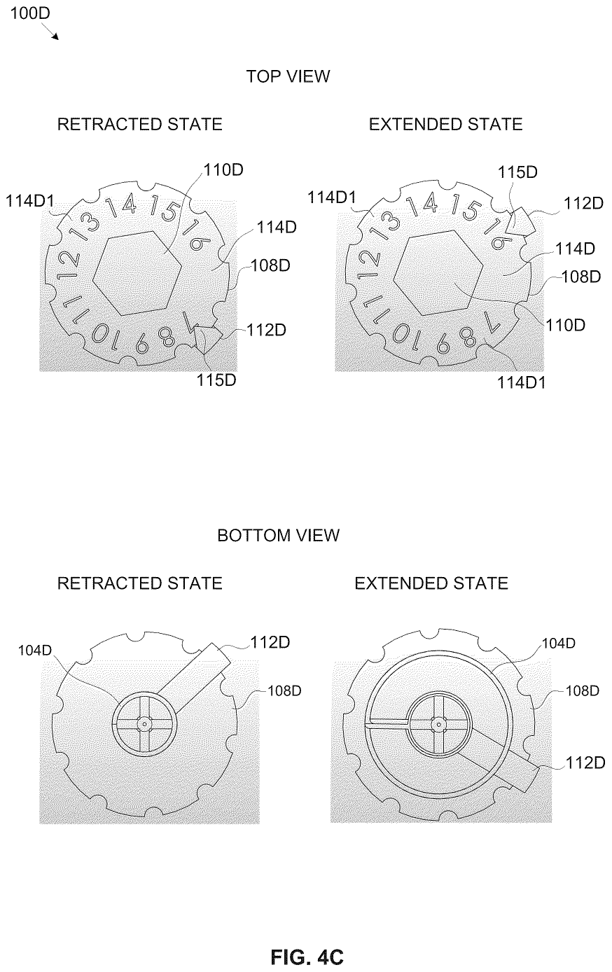

Reference is now made to FIG. 4A, FIG. 4B, and FIG. 4C, which are perspective views, front vies, side views, top views, and bottom views of a fourth exemplary embodiment of an apparatus having an adjustable diameter for forming a canal in bone medullary cavity in retracted and extended states, according to some embodiments of the present invention.

An exemplary apparatus 100D configured and operated to form a canal in the medullary cavity of one or more bones may comprise a shaft 102D, an actuator 106D and a laterally extendable assembly comprising an annular spring element 104D operatively coupled to the shaft 102D, specifically to a distal portion of the shaft 102C which is configured for insertion into the bone medullary cavity during the canal forming procedure.

The actuator 106D may comprise a joint assembly, specifically a first joint assembly 106D1 which is fixed relative to the shaft 102D in the longitudinal axis of the shaft 102D and a second joint assembly 106D2 moveable in the longitudinal axis of the shaft 102D.

The annular spring element 104D may transition between retracted and extended states according to a distance between the moveable second joint assembly 106D2 and the fixed first joint assembly 106D1. The level of extraction of the annular spring element 104D may define an external diameter 130 of the annular spring element 104D.

As seen, movement of the actuator 106D, specifically movement of the second joint assembly 106D2 may compress and/or release the annular spring element 104D in the lateral axis perpendicular to the longitudinal axis of the shaft 102D. movement of the second joint assembly 106D2 may therefore induce transition of the annular spring element 104D between (and including) a fully retracted state and a fully extended state, and hence define the external diameter 130 accordingly in a range between a minimal external diameter 130A in the fully retracted state and a maximal external diameter 130B in the fully extended state.

Moreover, when transitioned to the extended state, by movement of the actuator 106D, the annular spring element 104D may assume an at least slightly conic shape where the annular spring element 104D may be narrower at the distal end of the shaft 102 and wider at the proximal end of the shaft 102D. This is because the annular spring element 104D is fixedly connected to the distal end of the shaft 102D (via the first joint assembly 106D1) and may therefore extend to larger degree at the proximal end of the shaft 102D compared to the level of its extension at the distal end of the shaft 102D. The conic shape of the annular spring element 104D may improve penetration of the apparatus 100 into the medullary cavity.

Similarly to the apparatus 100A, movement of the second joint assembly 106D2 to set its distance from the first joint assembly 106D1 may be controlled via rotation and/or linear movement of a knob 108D such as the knob 108A. for example, in case of a threaded shaft 102D, the movement of the second joint assembly 106D2 may be controlled via a floating nut 106D3 such as the floating nut 106A3. Moreover, in such embodiments the knob 108D may comprise a polygon shaped socket (groove) 110D and/or a polygon shaped protrusion at a surface opposite the shaft 102D for attachment of a manual and/or electrically handle (power tool) configured to rotate the knob 108D.

Optionally, the apparatus 100D may comprise one or more locking elements 112D such as the locking elements 112A for locking the position of the moveable second joint assembly 106D2 to maintain the currently set external diameter 130 of the annular spring element 104D.

Optionally, the apparatus 100D may comprise a dial 114D such as the dial 114A and a pointer 115D indicative of the external diameter 130 of the annular spring element 104D. In order to illustrate the symbols, in this case digits of the dial 114D, the digit 13 which corresponds, for example, to an external diameter 130 of 13 mm is marked with numeral 114D1. However, to avoid clutter and maintain readability of the drawings, the other symbols (digits) of the dial 114D are not associated with numerals. The apparatus 100D may employ one or more structures, configurations and/or designs for implementing the dial 114D and the pointer 115D similarly to those used for the dial 104A and its pointer 115A. For example, the pointer 115D may be integrated with one or more of the locking elements 112D.

As stated for the apparatus 100C, while in the description of the apparatus 100C the first joint assembly 106D1 is fixed while the second joint assembly 106D2 is moveable, this should not be construed as limiting, since as may become apparent to a person skilled in the art the roles of the first and second joint assemblies 106D1 and 106D2 may be switched such that the second joint assembly 106D2 is fixed while the first joint assembly 106D1 is moveable. Moreover, other structures may be designed in which both the first and second joint assemblies 106D1 and 106D2 may be moveable with respect to each other.

Optionally, one or more sections of the annular spring element 104D may be shaped to have an outward bent edge, for example, a L shaped edge, and/or the like to improve extraction of bone marrow and formation of the medullary canal when the sections of the annular spring element 104D is rotated in the medullary cavity.

Optionally, one or more sections of the annular spring element 104D may be shaped to have a sharpened edge to further improve extraction of bone marrow and formation of the medullary canal when the sections of the annular spring element 104D is rotated in the medullary cavity.

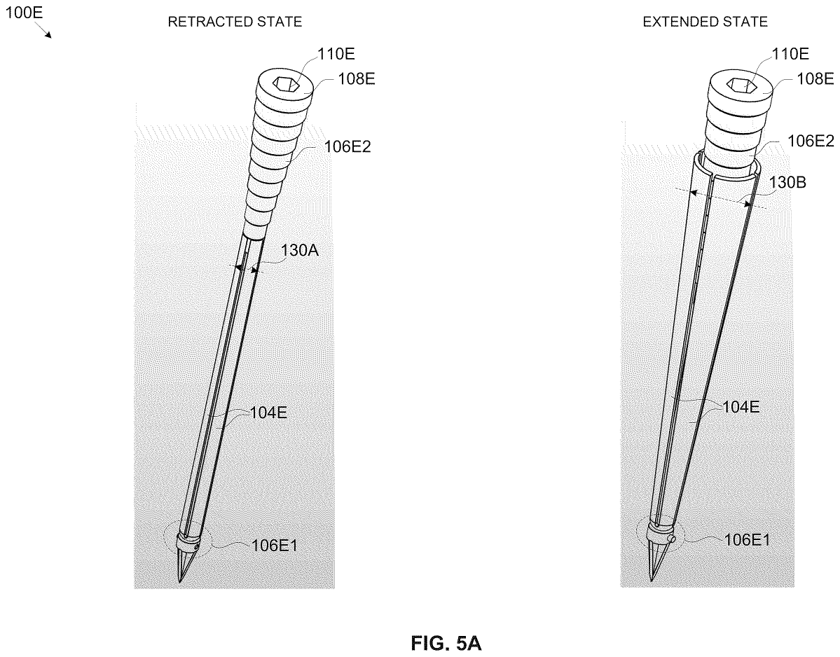

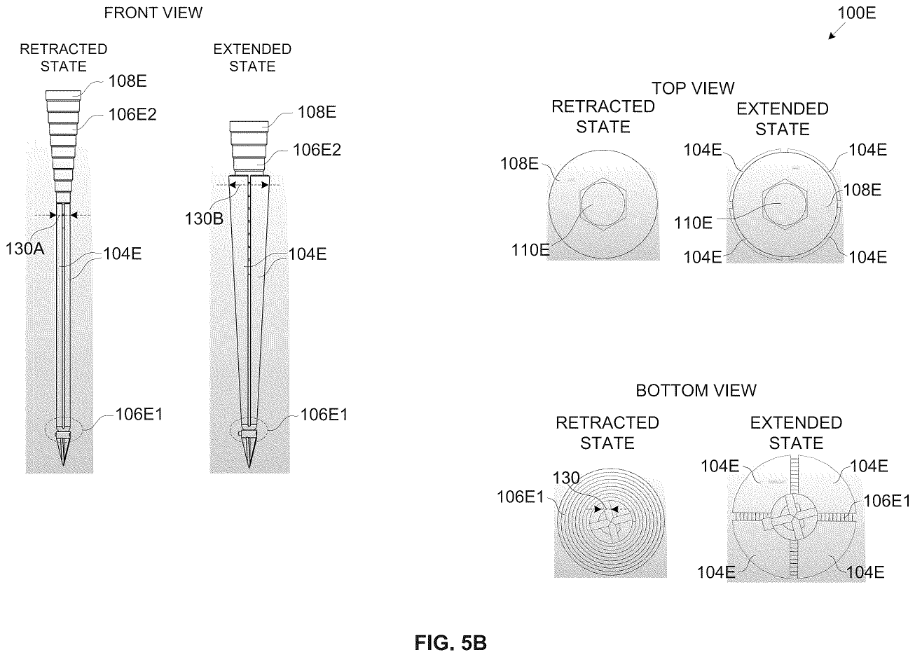

FIG. 5A, and FIG. 5B are perspective views, front views, side views, top views, and bottom views of a fourth exemplary embodiment of an apparatus having an adjustable diameter for forming a canal in bone medullary cavity in retracted and extended states, according to some embodiments of the present invention.

An exemplary apparatus 100E configured and operated to form a canal in the medullary cavity of one or more bones may comprise a shaft (not visible in the drawings), an actuator 106E and a laterally extendable assembly comprising a plurality of laterally extendable elements 104E, for example, four elements disposed along at least part of the shaft, for example, at a distal portion of the shaft which is configured for insertion into the bone medullary cavity during the canal forming procedure.

In particular, the plurality of laterally extendable elements 104E may be operatively coupled to the shaft via the actuator 106E which may comprise a conic piston 106E2 moveable relative to the shaft, specifically in the longitudinal axis of the shaft, and a joint assembly 106E1 mechanically coupling a distal end of the laterally extendable elements 104A to the shaft. At their proximal end, the laterally extendable elements 104E may be mechanically coupled to the conic piston

The conic piston 106E2 which is configured to be moveable along the longitudinal axis of the shaft may induce transition of the laterally extendable elements 104E between a retracted state and an extended state to adjust an external diameter 130 of the laterally extendable elements 104E.

As such, when the conic piston 106E2 is moved distally, i.e., downwards towards the joint assembly 106E1, the plurality of laterally extendable elements 104E may be pushed sideways thus increasing their external diameter 130. In contrast, when the conic piston 106E2 is moved proximally, i.e., upwards away from the joint assembly 106E1, the plurality of laterally extendable elements 104E may be pulled inward thus decreasing their external diameter 130.

The conic piston 106E2 may be moved to set the laterally extendable elements 104E in a plurality of states between (and including) a fully retracted state and a fully extended state which may define the external diameter 130 accordingly in a range between a minimal external diameter 130A and a maximal external diameter 130B.

The actuator 106E, specifically the conic piston 106E2 may be integrated with a knob 108E through which the conic piston 106E2 may be operated. For example, one or more knocking and/or pushing elements may be used to push the conic piston 106E 2 towards the joint assembly 106E1 and/or for pulling the conic piston 106E1 away from the joint assembly 106E1. For example, the knob 108E may comprise one or more sockets, grooves, protrusions and/or the like to support attachment of one or more knocking, pushing and/or rotation tools, for example, handle, a crowbar, a wrench, a ratchet, an Allen key, a hex key, a Torx key, and/or the like configured to knock, push, and/or rotate the knob 108E and the conic piston 106E2.

Moreover, the knob 108E may be used for attachment of one or more rotation tools, for example, handle, a wrench, a ratchet, an Allen key, a hex key, a Torx key, and/or the like configured for rotating the knob 108E and hence rotating the apparatus 100E, specifically the distal portion of the shaft and the laterally extendable elements 104E while inserted in the medullary cavity.

Optionally, the apparatus 100E may comprise one or more locking elements 112C such as the locking elements 112A for locking the conic piston 106E2 in place to limit its movement and prevent movement of the laterally extendable elements 104E to maintain their external diameter 130 in a fixed state.

Optionally, the apparatus 100E may comprise a dial 114C such as the dial 114A and a pointer indicative of the external diameter 130 of the laterally extendable elements 104C.

Optionally, one or more of the laterally extendable elements 104E may be shaped to have an outward bent edge, for example, a L shaped edge, and/or the like to improve extraction of bone marrow and formation of the medullary canal when the laterally extendable elements 104E are rotated in the medullary cavity.

Optionally, one or more of the laterally extendable elements 104E may be shaped to have a sharpened edge to further improve extraction of bone marrow and formation of the medullary canal when the laterally extendable elements 104C are rotated in the medullary cavity.

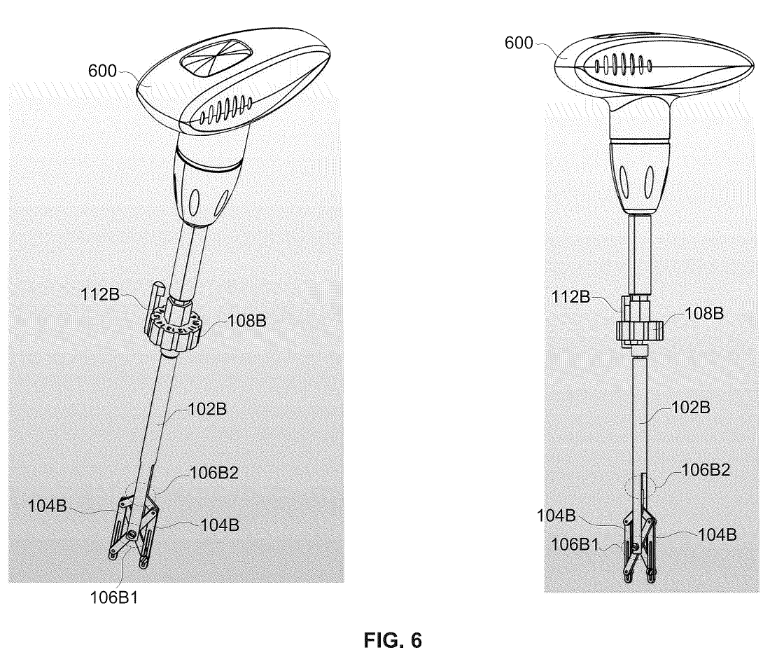

Reference is now made to FIG. 6, which presents perspective and side views of an exemplary handle attached to operate an apparatus having an adjustable diameter for forming a canal in bone medullary cavity in retracted and extended states, according to some embodiments of the present invention.

As described herein before, a medullary canal forming apparatus such as the apparatuses 100A, 100B, 100C, 100D, and/or 100E may comprise a knob 108A, 108B, 108C, 108D, and/or 108E respectively (collectively designated knob 108) configured for attachment of one or more tools 600, for example, handle, a crowbar, a wrench, a ratchet, an Allen key, a hex key, a Torx key, and/or the like configured to knock, push, pull, and/or rotate the knob 108 for operating an actuator such as the actuator 106A, 106B, 106C, 106D, and/or 106E respectively (collectively designated actuator 106) to induce transition of the a plurality of laterally extendable elements such as the elements 104A, 104B, 104C, 104D, and/or 104E respectively (collectively designated laterally extendable elements 104) to adjust their external diameter 130.

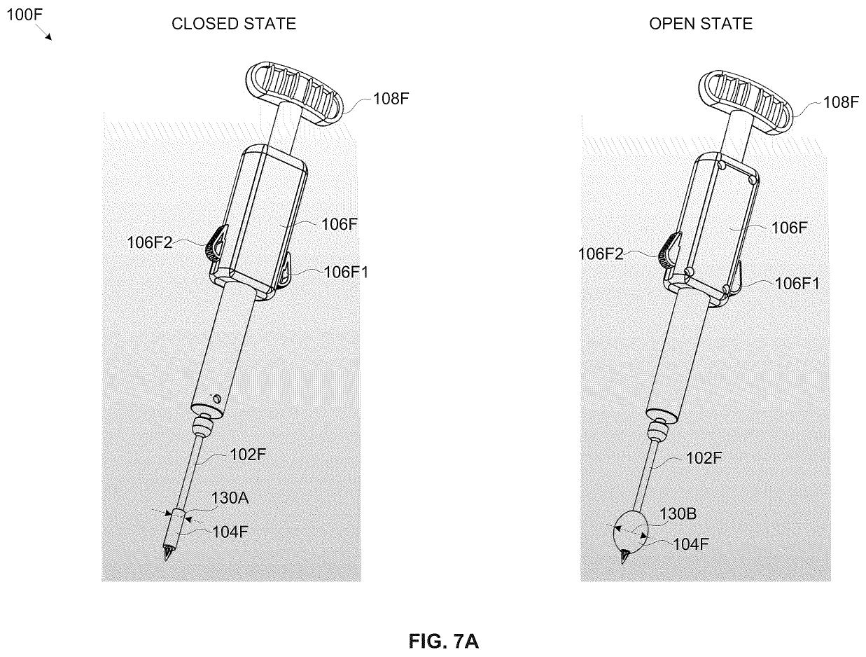

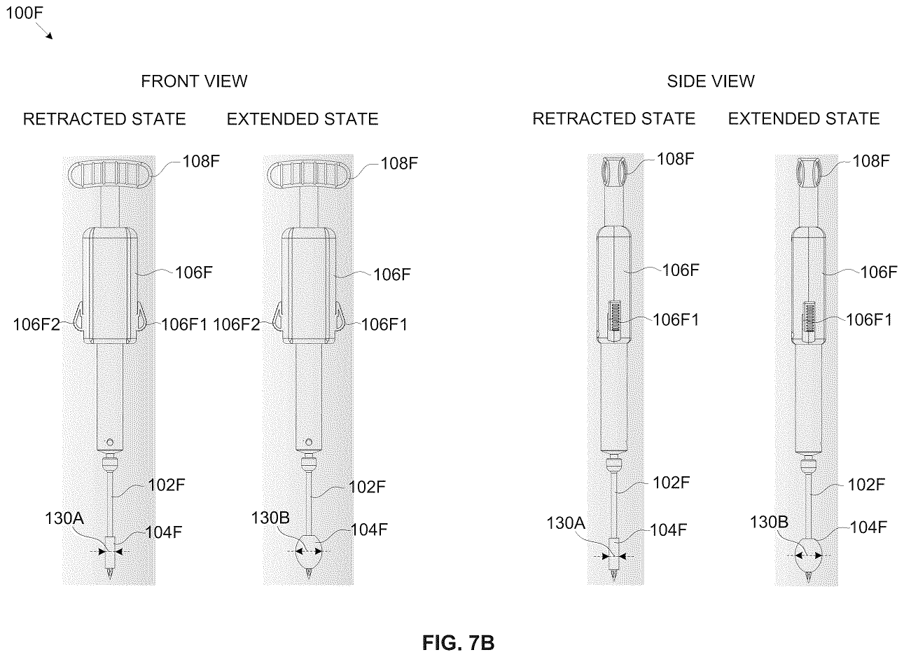

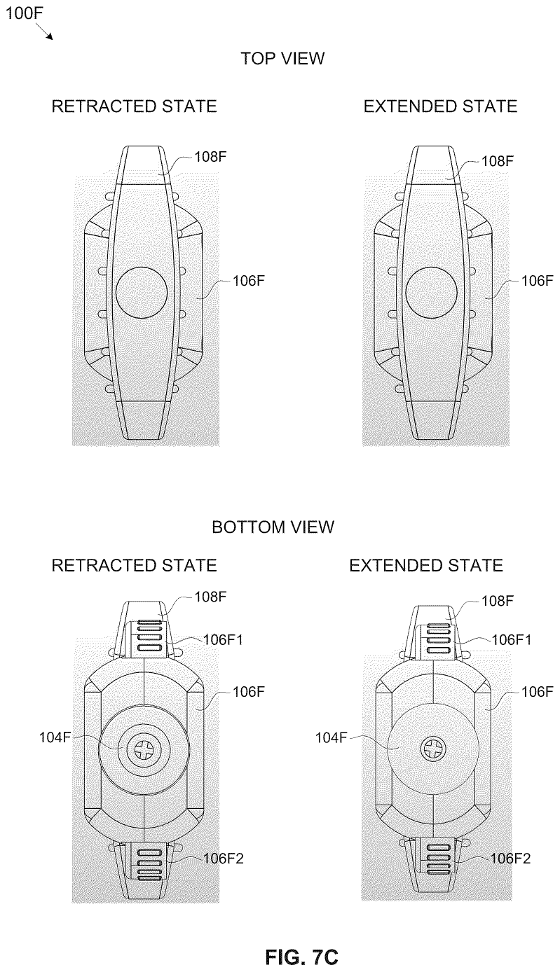

Reference is now made to FIG. 7A, FIG. 7B, and FIG. 7C, which are perspective views, front views, side views, top views, and bottom views of a sixth exemplary embodiment of an apparatus having an adjustable diameter for forming a canal in bone medullary cavity in retracted and extended states, according to some embodiments of the present invention.

An exemplary apparatus 100F configured and operated to form a canal in the medullary cavity of one or more bones may comprise a shaft 102F, an actuator 106F and a laterally extendable assembly comprising an inflatable element 104F disposed along at least part of the shaft, for example, at a distal portion of the shaft which is configured for insertion into the bone medullary cavity during the canal forming procedure.

In particular, the actuator 106F may comprise a pressure pump, for example, a manual pump, an electric pump, a hydraulic pump, and/or the like configured for inflating and deflating the inflatable element 104F to adjust an external diameter 130 of the inflatable element 104F. The actuator 106F may further include one or more pumping levers 106F, for example, a first pumping level 106F1 and a second pumping level 106F1 which may be used for manually operating the pressure pump to inflate and deflate the inflatable element 104F.

The actuator 106F may be operated to inflate and/or deflate the inflatable element 104F to adjust its external diameter in a plurality of states between a fully deflated state and a fully inflated which may define the external diameter 130 accordingly in a range between a minimal external diameter 130A and a maximal external diameter 130B.

Optionally, the actuator 106F may comprise a fluid, for example, liquid, gas (e.g. air), and/or the like inlet for connecting a pressure tube through which pressurized fluid may be pushed to inflate the inflatable element 104F and/or release fluid to deflate the inflatable element 104F.

The apparatus 100F may further include a knob 108F which may be operated to rotate the apparatus 100F, specifically the inflatable element 104F, for example, while the shaft 102F is inserted in the medullary cavity.

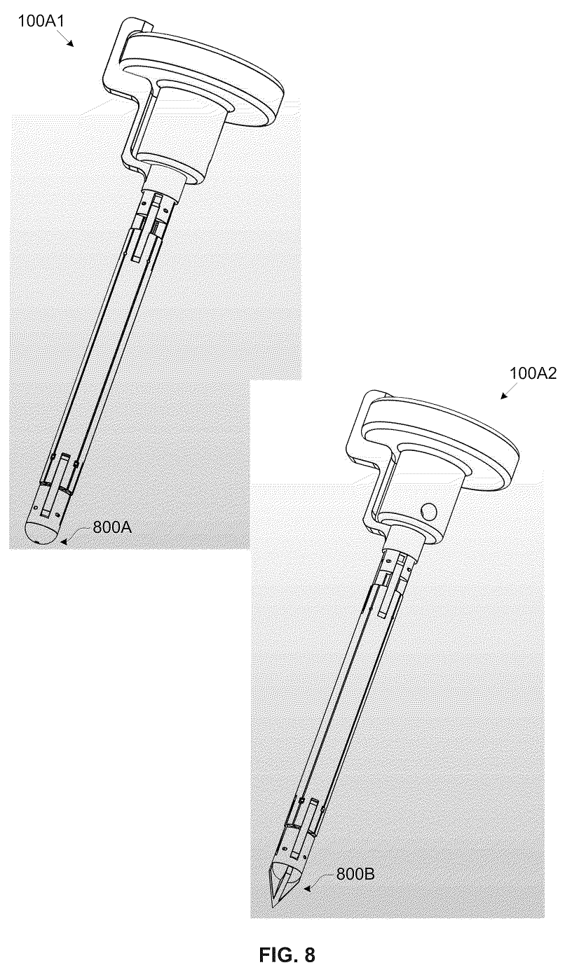

Reference is now made to FIG. 8, which presents perspective and side views of an exemplary apparatus having an adjustable diameter for forming a canal in bone medullary cavity with various end tips, according to some embodiments of the present invention.

As described herein before an apparatus for forming a canal in bones medullary cavity such as the apparatuses 100A, 100B, 100C, 100D, 100E, and/or 100F may be configured and/or shaped to have various tip types at the distal end of their shaft such as the shaft 102A, 102B, 102C, 102D, 102E, and/or 102F respectively, collectively designated shaft 102 herein after.

The distal tip of the shaft 102 may be shaped in a one or more of shapes to increase efficiency, robustness and/or reliability of the medullary canal forming. For example, the distal tip of the shaft 102 may be configured to have a rounded tip 800A. In another example, the distal tip of the shaft 102 may be configured to have a pointed tip 800B which may improve penetration of the apparatus 100 into the medullary cavity.

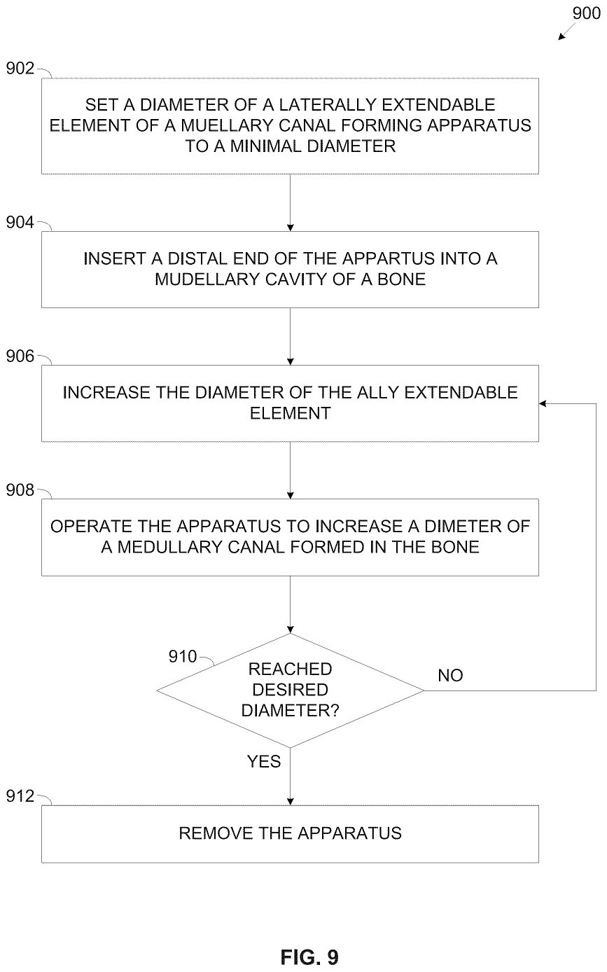

Reference is now made to FIG. 9, which is a flowchart of an exemplary process for using apparatus having an adjustable diameter to form a canal in a bone medullary cavity, according to some embodiments of the present invention.

An exemplary process 900 for forming a canal in a medullary cavity of a bone may be conducted during and/or in preparation for one or more medical and/or surgical procedures. For example, the process 900 may be conducted to form a medullary canal in the humerus bone in preparation for a shoulder joint replacement, for example, reverse shoulder, total shoulder, and/or the like in which a prosthesis is to be implanted in the humerus bone.

The process 900 may be conducted using an apparatus such as the apparatuses 100A, 100B, 100C, 100D, 100E, and/or 100F, collectively designated apparatus 100 herein after having a shaft 102, an actuator 106, and a laterally extendable element 104 having an external diameter 130 adjustable by movement of the actuator 106 relative to the shaft 102 which is controllable via a knob 108.

The designations 102, 104, 106, and 108 are applied to components which are generalizations of corresponding components in the embodiments described herein before. For example, the shaft 102 is a generalization of the shafts 102A, 102B, 102C, 102D, 102E, and 102F. In another example, the laterally extendable assembly 104 is a generalization of the laterally extendable elements 104A, 104B, 104C, 104D, 104E, and 104F. In another example, the actuator 106 is a generalization of the laterally actuators 106A, 106B, 106C, 106D, 106E, and 106F. In another example, the laterally extendable assembly 104 is a generalization of the laterally extendable elements 104A, 104B, 104C, 104D, 104E, and 104F. In another example, the knob 108 is a generalization of the knobs 108A, 108B, 108C, 108D, 108E, and 108F.

The process 900 is an iterative process in which the external diameter 130 of the laterally extendable assembly 104 is gradually increased in steps to gradually increase the diameter of the canal formed in the medullary cavity until reaching a desired canal diameter.

As shown at 902, the external diameter of the apparatus 100 may be set to a minimal diameter (value). In particular, the external diameter 130 of the portion (section) of the apparatus 100 which is intended to be inserted into the medullary cavity is set to its minimal diameter. The portion of the apparatus 100 which is inserted into the medullary cavity may include at least the laterally extendable assembly 104 and the shaft 102 and/or part thereof. The external diameter 130 therefore relates to the external diameter of the laterally extendable assembly 104.

To this end, a locking element such as, for example, the locking elements 112A, 112B, and/or 112C may be released to enable movement of an actuator 106 relative to the shaft 102.

While the locking element is in its released state, the actuator 106 may be operated via the knob 108 to move relative to the shaft 102 and induce transition of the laterally extendable assembly 104 to its most retracted and/or deflated state thus having the minimal external diameter 130.

Operating the knob 108 to move the actuator 106 may depend on the exact embodiment as described herein before. For example, in case of the apparatuses 100A, 100B, 100C, 100D and/or 100E, a tool, for example, a handle, a wrench, a key and/or the like may be attached to the knob 108 via the polygon socket, groove, and/or protrusion such as the polygon socket 110A, 110B, 110C, 110C and/or 110E respectively.

As shown at 904, the apparatus 100, specifically the distal portion of the apparatus 100 comprising the shaft 102 and the laterally extendable assembly 104 may be inserted into the medullary cavity of the bone to form a medullary canal in the medullary cavity.

Typically, the apparatus 100 may be inserted into a guide hole created in the medullary cavity after removing the bone top to uncover the medullary cavity.

Optionally, the apparatus 100 may be used to create the guide hole. For example, the apparatus 100 may be configured such that the range of the external diameter 130 of the laterally extendable assembly 104 may start from an extremely small diameter, for example, 4 mm, and/or 5 mm which is suitable for creating the guide hole.

As shown at 906, the external diameter 130 of the laterally extendable assembly 104 may be slightly increased. For example, assuming the initial minimal diameter was 5 mm, the external diameter 130 may be set to 6 mm. In another example, assuming the external diameter 130 set in a previous iteration is 11 mm, the external diameter 130 may be set to 12 mm.

Adjusting the external diameter 130 may be done by operating the knob 108 as described herein before while the lock element(s) are in their unlocked (released) state. For example, the handle attached to the knob may be operated, for example, rotated to move actuator 106 relative to the shaft 102 and adjust the diameter of the laterally extendable assembly 104 accordingly.

As shown at 908, while inserted in the medullary cavity, the apparatus 100 may be operated, for example, rotated, pushed, advanced, thrusted, knocked and/or the like to extract bone marrow and form the medullary canal in the bone. For example, in case of the apparatuses 100A, 100B, 100C, 100D and/or 100E, the tool attached to the knob 108 may be rotated to rotate the apparatus in the medullary cavity. In another example, in case of the apparatus 100F, the knob 108F may be rotated to rotate the apparatus in the medullary cavity.

Specifically, the apparatus 100 may be rotated while the locking element(s) is in the locked state thus limiting movement of the actuator 106 relative to the shaft 102 and preventing unwanted change in the external diameter 130 of the laterally extendable assembly 104.

As shown at 910, which is a conditional step, in case the desired canal diameter is reached the process may branch to 912. However, in case the diameter of the medullary canal is smaller than the desired diameter, the process may branch back to 906 to initiate another iteration in which the external diameter 130 of the laterally extendable assembly 104 may be further increased and the canal forming operation (step 908) is repeated.

As shown at 912, since the diameter of the medullary canal reached the desired diameter, the canal forming process may be ended and the apparatus 100 may be removed from within the medullary cavity of the bone.

Typically, before removing the apparatus 100 from the medullary cavity, the external diameter 130 of the laterally extendable assembly 104 may be reduced to enable smooth extraction of the apparatus 100 from within the medullary canal. For example, while the locking element(s) is in its released state, the actuator 106 may be operated and/or moved to adjust the external diameter 130 of the laterally extendable assembly 104 to the minimal diameter.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

As used herein the term “about” refers to ±10 %.

The terms “comprises”, “comprising”, “includes”, “including”, “having” and their conjugates mean “including but not limited to”. This term encompasses the terms “consisting of” and “consisting essentially of”.

The phrase “consisting essentially of” means that the composition or method may include additional ingredients and/or steps, but only if the additional ingredients and/or steps do not materially alter the basic and novel characteristics of the claimed composition or method.

As used herein, the singular form “a”, “an” and “the” include plural references unless the context clearly dictates otherwise. For example, the term “a compound” or “at least one compound” may include a plurality of compounds, including mixtures thereof.

The word “exemplary” is used herein to mean “serving as an example, an instance or an illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. An apparatus for forming a canal in a medullary cavity of a bone, comprising:
a shaft having a proximal portion and a distal portion;
an actuator configured to be moveable relative to the shaft, and
a laterally extendable assembly operatively coupled to the distal portion of the shaft, the laterally extendable assembly is configured to transition between retracted and extended states in a lateral axis perpendicular to a longitudinal axis of the shaft;
wherein a movement of the actuator relative to the shaft induces transition of the laterally extendable assembly between the retracted and extended states to adjust an external diameter of the laterally extendable assembly.

2. The apparatus of claim 1, wherein the external diameter is in a range of 4 to 20 millimeters.

3. The apparatus of claim 1, wherein the laterally extendable assembly is configured to transition between a plurality of discrete steps distributed between the retracted state and the extended state.

4. The apparatus of claim 1, wherein the laterally extendable assembly is configured to transition continuously between the retracted state and the extended state.

5. The apparatus of claim 1, further comprising a distal end of the shaft is pointed.

6. The apparatus of claim 1, further comprising a locking element configured to limit movement of the actuator with respect to the shaft and prevent change in extension and/or retraction of the laterally extendable assembly.

7. The apparatus of claim 1, further comprising a dial and a pointer mechanically connected to the actuator for indicating the external diameter.

8. The apparatus of claim 1, wherein the laterally extendable assembly comprises a plurality of laterally extendable elements disposed along at least part of the shaft and mechanically coupled to the shaft via the actuator, the actuator comprises a joint assembly having a plurality of rods which are tilt-able for retracting and extending the laterally extendable elements.

9. The apparatus of claim 8, wherein the joint assembly comprises a first joint assembly and a second joint assembly each comprising a plurality of rods, the first joint assembly is fixed in a longitudinal axis of the shaft and the second joint assembly is moveable in the longitudinal axis of the shaft, a tilt of the plurality of rods is proportional to a distance between the first joint assembly and the second joint assembly, the distance is adjustable by moving the second joint assembly in the longitudinal axis via a knob mounted at a proximal end of the shaft.

10. The apparatus of claim 9, wherein the shaft having a threaded exterior goes through a threaded bore of the first joint assembly and a threaded bore of the second joint assembly, the threaded shaft is rotatable via the knob such that rotation of the knob rotates the threaded shaft thus the moveable second joint assembly is moved in the longitudinal axis of the threaded shaft with respect to the fixed first joint assembly and the distance between the first joint assembly and the second joint assembly is adjusted accordingly.

11. The apparatus of claim 10, wherein the knob comprises a polygon shaped socket and/or a polygon shaped protrusion at a surface opposite the shaft for attachment of a rotation handle.

12. The apparatus of claim 9, wherein the shaft comprises an inner shaft disposed in a bore of an outer hollow shaft such that the inner shaft protrudes out of the outer shaft, the inner shaft is fixed to a fixed section of the knob while the outer shaft mechanically coupled to a second section of the knob is moveable in the longitudinal axis or vice versa, the first joint assembly is disposed on the fixed shaft and the second joint assembly is disposed on the moveable shaft, linear movement of the first section of the knob with respect to the second section of the knob moves the inner shaft with respect to the outer shaft in the longitudinal axis thus the moveable second joint assembly is moved in the longitudinal axis of the shaft with respect to the fixed first joint assembly and the distance between the first joint assembly and the second joint assembly is adjusted accordingly.

13. The apparatus of claim 8, further comprising at least one spring element configured to transition between compressed and released states, a tilt of the plurality of rods is proportional to compression of the at least one spring element.

14. The apparatus of claim 8, further comprising a member of a group consisting of:
at least one inflatable element configured to transition between inflated and deflated states, a tilt of the plurality of rods is proportional to inflation of the at least one inflatable element;
at least one of the plurality of laterally extendable elements is shaped to have an outward bent edge; and
at least one of the plurality of laterally extendable elements is shaped to have a sharpened edge.

15-16. (canceled)

17. The apparatus of claim 1, wherein the laterally extendable assembly comprises an annular spring element disposed along at least part of the shaft and mechanically coupled to the shaft via the actuator, the actuator comprises a joint assembly comprising a first joint assembly fixed in a longitudinal axis of the shaft and a second joint assembly moveable in the longitudinal axis of the shaft, extension and retraction of the annular spring element is proportional to a distance between the first joint assembly and the second joint assembly, the distance is adjustable by moving the second joint assembly in the longitudinal axis via a knob mounted at a proximal end of the shaft.

18. The apparatus of claim 1, wherein the laterally extendable assembly comprises a plurality of laterally extendable elements disposed along at least part of the shaft and mechanically coupled to the shaft via the actuator which comprises a conic piston moveable along a longitudinal axis of the shaft for inducing transition of the laterally extendable assembly between a retracted state and an extended state to adjust an external diameter of the laterally extendable assembly.

19. A method of forming a medullary canal in a bone, comprising:
configuring an external diameter of a laterally extendable assembly of a medullary canal forming apparatus to a minimal value, the external diameter is defined by an actuator moveable relative to a shaft of the apparatus for transitioning the laterally extendable assembly between a retracted and extended states;
inserting a distal portion of the apparatus into a guide hole created in a medullary cavity of a bone to form a canal in the bone;
increasing the external diameter by moving the actuator relative to the shaft to extend the laterally extendable assembly;
rotating the shaft to increase a dimeter of the canal;
gradually increasing the external diameter and rotating the shaft to increase the diameter of the canal until reaching a desired diameter;
reducing the external diameter by moving the actuator relative to the shaft to retract the laterally extendable assembly; and
removing the apparatus from the medullary cavity.

20. The method of claim 19, wherein the medullary canal is formed for insertion of a prosthesis implant.

21. The method of claim 19, further comprising using the apparatus to create the guide hole in the medullary cavity.

22. An apparatus for forming a canal in a medullary cavity of a bone, comprising:
a shaft having a proximal portion and a distal portion;
a laterally extendable assembly operatively coupled to the distal portion of the shaft, the laterally extendable assembly comprises an inflatable element; and
an actuator configured for inflating and deflating the inflatable element to adjust an external diameter of the inflatable element.
